(19) United States
(12) Patent Application Publication
(10) Pub. No.: US 2025/0228465 A1
(43) Pub. Date: Jul. 17, 2025

(54) BIOLOGICAL INFORMATION DETECTION DEVICE AND VEHICLE INCLUDING SAME

(71) Applicant: Murata Manufacturing Co., Ltd., Nagaokakyo-shi (JP)

(72) Inventor: Ryo SAITO, Nagaokakyo-shi (JP)

(73) Assignee: Murata Manufacturing Co., Ltd., Nagaokakyo-shi (JP)

(21) Appl. No.: 19/171,360

(22) Filed: Apr. 7, 2025

Related U.S. Application Data
(63) Continuation of application No. PCT/JP2023/041364, filed on Nov. 16, 2023.

(30) Foreign Application Priority Data
Dec. 7, 2022 (JP) 2022-195471

(51) Int. Cl. A61B 5/05; A61B 5/00; B60N 2/00

(52) U.S. Cl. CPC A61B 5/05; A61B 5/6893; A61B 5/721; B60N 2/0022; B60N 2/0027; B60N 2/0031; B60N 2/0035; A61B 2503/22; A61B 2562/04; B60N 2210/20; B60N 2220/00; B60N 2230/30

(57) ABSTRACT

To realize a biological information detection device capable of obtaining accurate biological information. The biological information detection device includes a radio wave sensor that detects a body surface displacement of a human body; a first vibration sensor that is disposed together with the radio wave sensor at a position away from the supporting surface that supports the human body; a second vibration sensor that is disposed closer to the supporting surface than the first vibration sensor; and a signal processing unit that generates a biosignal of the human body based on the body surface displacement obtained by the radio wave sensor, a first vibration component obtained by the first vibration sensor, and a second vibration component obtained by the second vibration sensor.

### CROSS REFERENCE TO RELATED APPLICATIONS

This is a continuation application of PCT/JP2023/041364, filed on Nov. 16, 2023, designating the United States of America, which is based on and claims priority to Japanese Patent Application No. JP 2022-195471 filed on Dec. 7, 2022. The entire contents of the above-identified applications, including the specifications, drawings and claims, are incorporated herein by reference in their entirety.

### TECHNICAL FIELD

The present disclosure relates to a biological information detection device and a vehicle including the same.

### BACKGROUND ART

There is a known occupant state detection system that detects occupants in a driver's seat, a front passenger seat, and back seats, and for the driver's seat, also obtains biological information in addition to detecting an occupant (see, for example, Patent Document 1). In the occupant state detection system of Patent Document 1, a radio wave sensor installed in a vehicle transmits radio waves and receives reflected waves of the radio waves to detect distances from reflecting objects. The occupant state detection system calculates distance variations based on the distances to the reflecting objects detected over time and thereby determines whether the detected distances are changing. Assuming there is no distance variation or the distance variations are less than or equal to a predetermined value, it is determined that there is no person (or occupant) in the vehicle. Also, assuming there is a distance variation or assuming a distance variation is greater than the predetermined value, it is determined that a person exists in the vehicle. However, assuming the radio wave sensor vibrates or an object, such as a person, vibrates, a vibration component resulting from the vibration is included in a radio wave sensor signal output by the radio wave sensor. Assuming the vibration component is included in the radio wave sensor signal, the detection accuracy of a signal processing system may decrease.

For example, a signal processing system and a sensor system are disclosed that can improve the accuracy in detecting a state of an object by attenuating a vibration component in a radio wave sensor signal (see, for example, Patent Document 2). The signal processing system of Patent Document 2 includes a first reception unit, a second reception unit, and a signal processing unit. The first reception unit receives a radio wave sensor signal from a radio wave sensor that receives a radio wave reflected by an object. The second reception unit receives, from a vibration sensor, a vibration sensor signal corresponding to the vibration of at least one of the radio wave sensor and the object. The signal processing unit detects information regarding the state of the object based on the radio wave sensor signal and the vibration sensor signal.

### CITATION LIST

### Patent Documents

Patent Document 1: Japanese Unexamined Patent Application Publication No. 2018-202921
Patent Document 2: Japanese Unexamined Patent Application Publication No. 2021-071326

### SUMMARY OF DISCLOSURE

### Technical Problem

In the signal processing system and the sensor system disclosed in Patent Document 1, because the radio wave sensor (radar) is a sensor that measures a distance, the vibration mixed into the measurement value is the vibration of a human body with reference to the installation position of the radio wave sensor. Because the human body is on a material with elasticity, such as urethane, the human body vibrates differently from the vibration of the vehicle (for example, automobile) itself. That is, the measurement value of the radar is influenced by, in addition to the biological information of a measurement target (human body), a movement of the human body associated with the driving of the vehicle. With the related-art technology described above, it is possible to suppress the influence of the movement of the human body associated with the driving of the vehicle by using the measurement value of the vibration sensor. However, the measurement value of the vibration sensor includes vibrations originating from the vibrations of the body of the vehicle in addition to the movement of the human body associated with the driving of the vehicle. This in turn may reduce the accuracy of biological information of a human body, which is a measurement target.

The present disclosure has been made in view of the above problem, and an object of the present disclosure is to realize a biological information detection device capable of obtaining accurate biological information and a vehicle including the biological information detection device.

### Solution to Problem

A biological information detection device according to an aspect of the present disclosure includes a radio wave sensor that detects a body surface displacement of a human body; a first vibration sensor that is disposed together with the radio wave sensor at a position away from a supporting surface that supports the human body; a second vibration sensor that is disposed closer to the supporting surface than the first vibration sensor; and a signal processing unit that generates a biosignal of the human body based on the body surface displacement obtained by the radio wave sensor, a first vibration component obtained by the first vibration sensor, and a second vibration component obtained by the second vibration sensor.

With this configuration, the biosignal of the human body is generated based on the body surface displacement obtained by the radio wave sensor, the first vibration component obtained by the first vibration sensor, and the second vibration component obtained by the second vibration sensor. This configuration makes it possible to suppress both the influence of a body movement component of a subject and the influence of vibration components resulting from vibrations of the body of a vehicle (e.g., an automobile).

A vehicle according to an aspect of the present disclosure includes the biological information detection device described above.

This configuration makes it possible to realize a vehicle including a biological information detection device that is capable of suppressing both the influence of a body movement component of a subject and the influence of vibration components resulting from vibrations of the body of a vehicle and thereby obtaining accurate biological information.

### Advantageous Effects of Disclosure

The present disclosure makes it possible to realize a biological information detection device capable of obtaining accurate biological information and a vehicle including the biological information detection device.

### BRIEF DESCRIPTION OF DRAWINGS

FIG. 1 is a side view of a biological information detection device according to a first embodiment, which is used for a driver monitoring system of a vehicle.

FIG. 2 is a block diagram illustrating a schematic configuration of the biological information detection device according to the first embodiment.

FIG. 3A is a diagram showing an example of a relationship between a body surface displacement obtained by a radio wave sensor and a first vibration component obtained by a first vibration sensor.

FIG. 3B is a diagram showing an example of a relationship between a first vibration component obtained by a first vibration sensor and a second vibration component obtained by a second vibration sensor.

FIG. 3C is a diagram showing an example of a relationship between a body surface displacement obtained by a radio wave sensor and an estimated vibration component generated by a vibration estimation unit.

FIG. 4 is a side view of a biological information detection device according to a second embodiment, which is used for a driver monitoring system of a vehicle.

FIG. 5 is a block diagram illustrating a schematic configuration of the biological information detection device according to the second embodiment.

FIG. 6 is a diagram for describing an example of a coordinate rotation method according to the second embodiment.

FIG. 7 is a side view of a biological information detection device according to a third embodiment, which is used for a driver monitoring system of a vehicle.

FIG. 8 is a block diagram illustrating a schematic configuration of the biological information detection device according to the third embodiment.

### DESCRIPTION OF EMBODIMENTS

A biological information detection device and a vehicle including the biological information detection device according to embodiments are described below with reference to the drawings. However, the present disclosure is not limited to the embodiments described below.

### First Embodiment

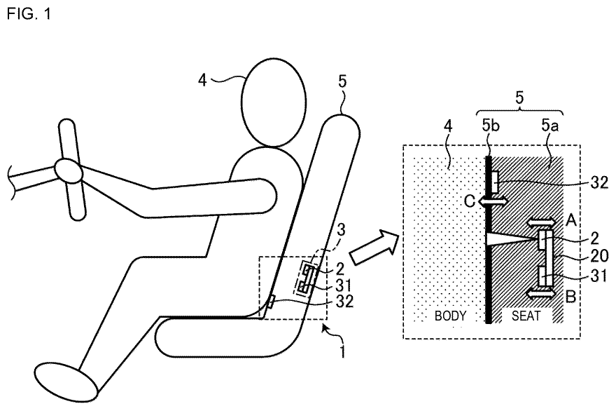

FIG. 1 is a side view of a biological information detection device according to a first embodiment, which is used for a driver monitoring system of a vehicle. A biological information detection device 1 is used for, for example, a driver monitoring system (DMS) illustrated in FIG. 1, and is disposed inside of a seat 5 on which a driver, who is a subject 4, is seated in the vehicle. As illustrated in FIG. 1, the biological information detection device 1 includes a radio wave sensor 2, a first vibration sensor 31, and a second vibration sensor 32.

As shown by an enlarged view of a part of FIG. 1 enclosed by a dotted line, the biological information detection device 1 is disposed inside of the seat 5. Specifically, the biological information detection device 1 is disposed inside of an internal material 5a of the seat 5. For example, the first vibration sensor 31 may be disposed on a frame of the seat 5 together with the radio wave sensor 2.

The radio wave sensor 2 is, for example, a radar device provided on a dielectric substrate 20. The radio wave sensor 2 is disposed near the back side of the seat 5. The radio wave sensor 2 detects a variation in the distance to the body surface (body surface displacement) of the subject 4 to which an electromagnetic wave is emitted.

Examples of materials for the dielectric substrate 20 include a low temperature co-fired ceramics (LTCC) multilayer substrate; a multilayer resin substrate formed by laminating multiple resin layers composed of resins, such as epoxy and polyimide; a multilayer resin substrate formed by laminating multiple resin layers composed of a liquid crystal polymer (LCP) with a lower permittivity; a multilayer resin substrate formed by laminating multiple resin layers composed of a fluorine resin; and a ceramic multilayer substrate (other than a low temperature co-fired ceramics multilayer substrate).

A transceiving antenna (not shown) of the radio wave sensor 2 is provided on a surface of the dielectric substrate 20 that faces the body surface of the subject 4. The radio wave sensor 2 emits an electromagnetic wave toward the body surface of the subject 4 from the transceiving antenna provided on the dielectric substrate 20 and receives the electromagnetic wave reflected from the body surface of the subject 4 via the transceiving antenna. The electromagnetic wave is modulated by the radio wave sensor 2 according to, for example, a Doppler method, a frequency modulated continuous wave (FMCW) radar method, or a pulse modulation scheme. In addition to the modulation schemes described above, any other modulation scheme, which enables the measurement of a variation in the distance to the body surface of the subject 4 or a target object, may be used by the radio wave sensor 2 to modulate an electromagnetic wave. In the descriptions below, the surface of the dielectric substrate 20, on which the transceiving antenna is provided, is also referred to as an “antenna surface”.

The first vibration sensor 31 and the second vibration sensor 32 are, for example, acceleration sensors. The first vibration sensor 31 is disposed near the back side of the seat 5 together with the radio wave sensor 2. For example, the first vibration sensor 31 is disposed on the dielectric substrate 20, on which the radio wave sensor 2 is provided, and is placed in a housing 3 together with the radio wave sensor 2. The second vibration sensor 32 is disposed near the front side of the seat 5. Specifically, the second vibration sensor 32 is disposed on the back side of a surface material 5b of, for example, the backrest or the seat base of the seat 5, which contacts the body of the subject 4, that is, disposed in the internal material 5a of the seat 5 at a position closer to the surface material 5b. In other words, the first vibration sensor 31 is disposed, together with the radio wave sensor 2, at a position away from the supporting surface (for example, the back side of the surface material 5b of the seat 5) for supporting the human body, and the second vibration sensor 32 is disposed closer to the supporting surface for supporting the human body than the first vibration sensor 31.

In the present disclosure, the biological information detection device 1 detects biological information (vital signs), such as the heart rate, heart rate variability, respiratory rate, and depth of breathing of the subject 4, who is driving the vehicle, based on a body surface displacement (a variation in the distance to the body surface of the subject 4) obtained by the radio wave sensor 2 and variations in vibrations obtained by the first vibration sensor 31 and the second vibration sensor 32. In the present embodiment, it is assumed that a direction A of the body surface displacement obtained by the radio wave sensor 2, a vibration detection direction (acceleration detection direction) B of the first vibration sensor 31, and a vibration detection direction C of the second vibration sensor 32 are the same.

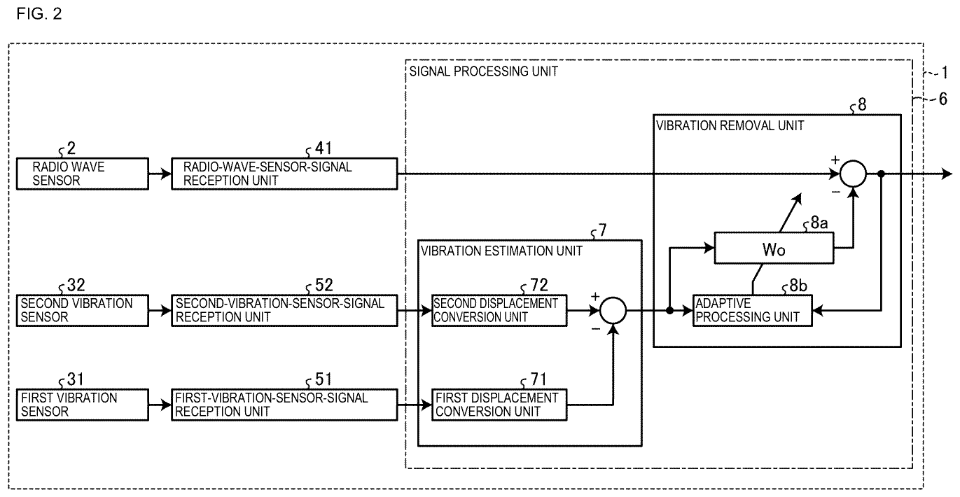

FIG. 2 is a block diagram illustrating a schematic configuration of the biological information detection device according to the first embodiment. The biological information detection device 1 according to the first embodiment includes, in addition to the radio wave sensor 2, the first vibration sensor 31, and the second vibration sensor 32, a radio-wave-sensor-signal reception unit 41, a first-vibration-sensor-signal reception unit 51, a second-vibration-sensor-signal reception unit 52, and a signal processing unit 6.

For example, the radio-wave-sensor-signal reception unit 41, the first-vibration-sensor-signal reception unit 51, the second-vibration-sensor-signal reception unit 52, and the signal processing unit 6 are implemented as modules that are provided on the back side of the antenna surface of the dielectric substrate 20. Each of these units may be implemented by a software control process performed by a microcomputer, by a hardware configuration of an electronic circuit, or by both of a software control process performed by a microcomputer and a hardware configuration of an electronic circuit. Specifically, the signal processing unit 6 is implemented as, for example, an integrated circuit (IC).

The radio wave sensor 2 generates, for example, a continuous wave (CW) signal as a transmission wave and transmits the transmission wave toward the body of the subject 4. For example, in a configuration in which a Doppler method is used as the modulation scheme for modulating an electromagnetic wave by the radio wave sensor 2, the radio wave sensor 2 outputs a radio wave sensor signal, which corresponds to a difference in frequency between a transmission wave and a reflected wave that are transmitted and received, to the radio-wave-sensor-signal reception unit 41. The transmission wave may be, for example, either a millimeter wave or a microwave. In the example of the present embodiment, the radio wave sensor 2 emits an electromagnetic wave (e.g., a millimeter wave) as a transmission wave. However, the transmission wave may be any other type of wave, such as a sound wave or a light wave.

The radio-wave-sensor-signal reception unit 41 receives a radio wave sensor signal from the radio wave sensor 2, amplifies the radio wave sensor signal as appropriate, converts the amplified radio wave sensor signal into a digital signal with an AD converter, then converts the digital signal into a displacement signal, and outputs the displacement signal as a body surface displacement signal to the signal processing unit 6.

The first vibration sensor 31 outputs a first vibration sensor signal corresponding to a detected vibration to the first-vibration-sensor-signal reception unit 51.

The first-vibration-sensor-signal reception unit 51 receives the first vibration sensor signal from the first vibration sensor 31, amplifies the first vibration sensor signal as appropriate, converts the amplified first vibration sensor signal into a digital signal with an AD converter, and outputs the digital signal to the signal processing unit 6.

The second vibration sensor 32 outputs a second vibration sensor signal corresponding to a detected vibration to the second-vibration-sensor-signal reception unit 52.

The second-vibration-sensor-signal reception unit 52 receives the second vibration sensor signal from the second vibration sensor 32, amplifies the second vibration sensor signal as appropriate, converts the amplified second vibration sensor signal into a digital signal with an AD converter, and outputs the digital signal to the signal processing unit 6.

The signal processing unit 6 includes a vibration estimation unit 7 and a vibration removal unit 8.

The vibration estimation unit 7 includes a first displacement conversion unit 71 and a second displacement conversion unit 72. The first displacement conversion unit 71 converts the first vibration sensor signal output from the first-vibration-sensor-signal reception unit 51 into a first vibration signal. The first vibration signal is a first vibration component obtained by the first vibration sensor 31. The second displacement conversion unit 72 converts the second vibration sensor signal output from the second-vibration-sensor-signal reception unit 52 into a second vibration signal. The second vibration signal is a second vibration component obtained by the second vibration sensor 32. The vibration estimation unit 7 outputs an estimated vibration component, which is obtained by subtracting the first vibration component from the second vibration component, to the vibration removal unit 8 as an estimated vibration signal.

The vibration removal unit 8 separates the estimated vibration signal, which is a calculation result of the vibration estimation unit 7, from the body surface displacement signal output from the radio-wave-sensor-signal reception unit 41 and thereby generates a biosignal of the subject 4. As a result, a biosignal, from which unnecessary vibration components have been separated, is obtained.

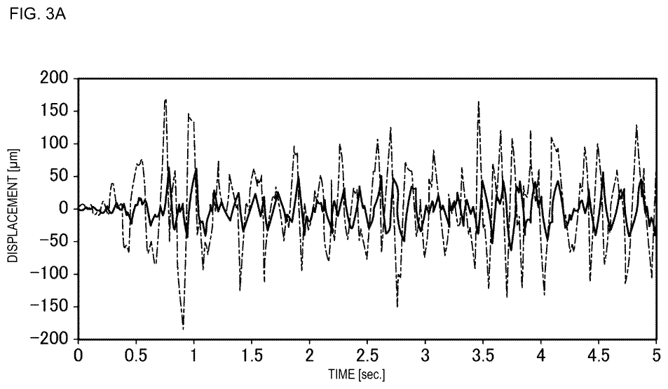

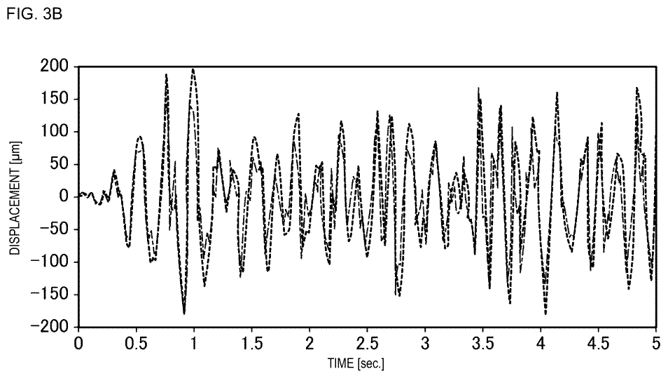

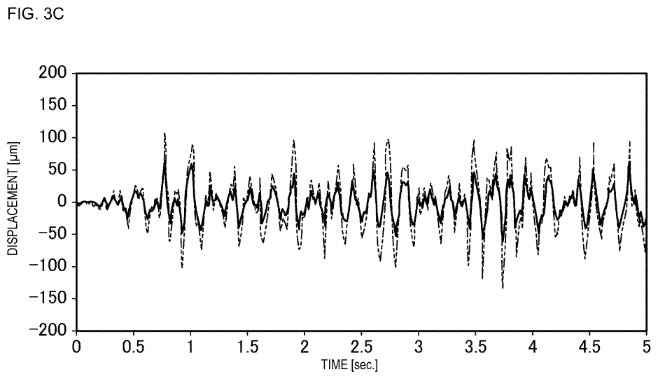

Here, a concept of obtaining accurate biological information in the biological information detection device 1 according to the present disclosure is described. FIG. 3A is a diagram showing an example of a relationship between a body surface displacement obtained by the radio wave sensor and a first vibration component obtained by the first vibration sensor. FIG. 3B is a diagram showing an example of a relationship between a first vibration component obtained by the first vibration sensor and a second vibration component obtained by the second vibration sensor. FIG. 3C is a diagram showing an example of a relationship between a body surface displacement obtained by the radio wave sensor and an estimated vibration component generated by the vibration estimation unit. In each of FIGS. 3A, 3B, and 3C, the horizontal axis indicates time, and the vertical axis indicates a displacement. Also, in each of FIGS. 3A, 3B, and 3C, a solid line indicates a body surface displacement obtained by the radio wave sensor 2, a dotted line indicates a second vibration component obtained by the second vibration sensor 32, a dashed-dotted line indicates a first vibration component obtained by the first vibration sensor 31, and a dashed-two dotted line indicates an estimated vibration component generated by the vibration estimation unit 7. Here, in each of FIGS. 3A, 3B, and 3C, the direct current component of each component is omitted.

A body surface displacement (the solid line in FIG. 3A) obtained by the radio wave sensor 2 includes, in addition to a displacement component (hereafter also referred to as a “biosignal”) resulting from biological information of the subject 4, who is the detection target of the biological information detection device 1 of the present disclosure, a vibration component (hereafter also referred to as a “body movement component”) associated with a body movement of the subject 4 (a movement of the body of the subject 4 associated with the driving of the vehicle).

The vibration removal unit 8 generates a biosignal of the subject 4 by separating the body movement component from the body surface displacement obtained by the radio wave sensor 2 (hereafter also referred to as “adaptive processing”) by using an adaptive filter described later.

The second vibration component (the dotted line in FIG. 3B) obtained by the second vibration sensor 32 includes, in addition to the body movement component of the subject 4, vibration components resulting from a vibration associated with the behavior of the body of a vehicle (for example, an automobile) and a vibration of an engine (which are hereafter also referred to as “vehicle body vibrations”). On the other hand, the first vibration component (the dashed-dotted line in FIGS. 3A and 3B) obtained by the first vibration sensor 31 includes many vibration components resulting from the vehicle body vibrations.

In the present disclosure, as described above, the vibration estimation unit 7 generates the estimated vibration component by subtracting the first vibration component obtained by the first vibration sensor 31 from the second vibration component obtained by the second vibration sensor 32. This makes it possible to obtain an estimated vibration component with reduced influence of vibration components resulting from vehicle body vibrations.

Thus, a vibration signal (the dashed-two dotted line in FIG. 3C) mainly composed of the body movement component of the subject 4 is generated by subtracting the first vibration component (the dashed-dotted line in FIG. 3B), which is obtained by the first vibration sensor 31 and includes many vibration components resulting from vehicle body vibrations, from the second vibration component (the dotted line in FIG. 3B), which is obtained by the second vibration sensor 32 and includes vibration components resulting from vehicle body vibrations in addition to the body movement component of the subject 4. Accurate biological information can be obtained by performing adaptive processing using this vibration signal.

Specifically, the vibration removal unit 8 separates the estimated vibration signal calculated by the vibration estimation unit 7 from the body surface displacement signal output from the radio-wave-sensor-signal reception unit 41 by using, for example, an adaptive filter that uses an algorithm, such as Least Mean Square (LMS) or Recursive Least Square (RLS). As a result, a biosignal, from which unnecessary vibration components have been separated, is obtained.

The method for separating a vibration sensor signal from a radio wave sensor signal is not limited to the example described above. Other examples include a method using Blind Source Separation (BBS), such as Independent Component Analysis (ICA), Independent Vector Analysis (IVA), or Independent Low-Rank Matrix Analysis (ILRMA); and a method using mode decomposition, such as Ensemble Empirical Mode Decomposition (EEMD) or Multivariate Variational Mode Decomposition (MVMD). The present disclosure is not limited by the method for separating a vibration sensor signal from a radio wave sensor signal.

In the configuration illustrated in FIG. 2, an adaptive processing unit 8b can variably set a filter coefficient Wo of a filter 8a. The filter 8a generates an estimated vibration signal by convolving a vibration sensor signal with the filter coefficient Wo. The estimated vibration signal corresponds to an estimation result of vibration components included in the body surface displacement signal obtained by the radio wave sensor 2. The vibration removal unit 8 generates a filtering signal as a biosignal by subtracting the estimated vibration signal from the body surface displacement signal.

As described above, the body surface displacement signal obtained by the radio wave sensor 2 includes a body movement component of the subject 4 (a movement component of the body of the subject 4 associated with the driving of a vehicle) in addition to a biosignal of the body of the subject 4. The adaptive processing unit 8b sets the filter coefficient Wo such that the correlation between the vibration sensor signal and the filtering signal (the biosignal) is minimized. This makes it possible to obtain a biosignal (filtering signal) by attenuating the body movement component of the subject 4 in the radio wave sensor signal.

By using biosignals obtained as described above to obtain vital signs of the body of the subject 4, it is possible to suppress both the influence of the body movement component of the subject 4 and the influence of the vibration components resulting from vehicle body vibrations and thereby obtain accurate biological information. Details of the method of obtaining vital signs are omitted here, and the present disclosure is not limited by the method of obtaining vital signs.

According to the biological information detection device 1 of the first embodiment, the first vibration component, which is obtained by the first vibration sensor 31 and includes many vibration components resulting from vehicle body vibrations, is subtracted from the second vibration component, which is obtained by the second vibration sensor 32 and includes vibration components resulting from vehicle body vibrations in addition to the body movement component of the subject 4. As a result, an estimated vibration component with reduced influence of vibration components resulting from vehicle body vibrations is obtained. Then, the estimated vibration signal is separated from the body surface displacement signal obtained by the radio wave sensor 2 to generate a human biosignal in which the body movement component of the subject 4 is suppressed. This makes it possible to obtain accurate biological information.

### Second Embodiment

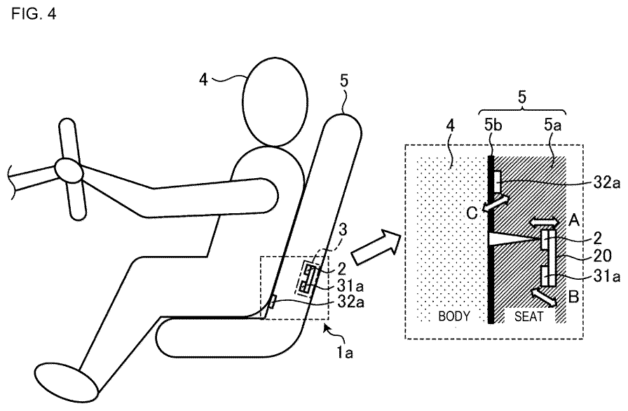

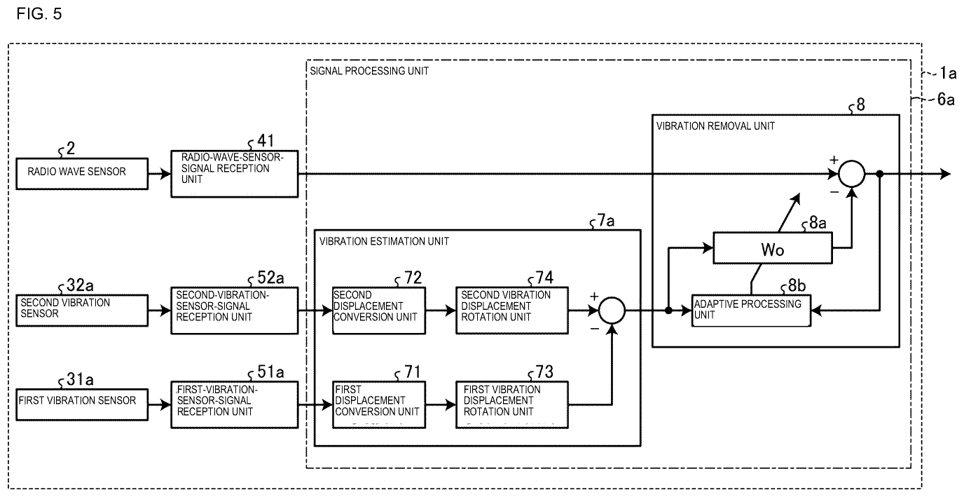

FIG. 4 is a side view of a biological information detection device according to a second embodiment, which is used for a driver monitoring system of a vehicle. FIG. 5 is a block diagram illustrating a schematic configuration of the biological information detection device according to the second embodiment. In FIG. 4, the housing 3 is omitted.

In a biological information detection device 1a according to the second embodiment, each of a first vibration sensor 31a and a second vibration sensor 32a is a triaxial acceleration sensor. In the present embodiment, a direction A of displacement detected by the radio wave sensor 2, a vibration detection direction (acceleration detection direction) B of the first vibration sensor 31, and a vibration detection direction C of the second vibration sensor 32 are different from each other.

The first vibration sensor 31a outputs a first vibration sensor signal corresponding to a detected vibration in a triaxial direction to a first-vibration-sensor-signal reception unit 51a.

The first-vibration-sensor-signal reception unit 51a receives the first vibration sensor signal corresponding to the vibration in the triaxial direction from the first vibration sensor 31a, amplifies the first vibration sensor signal as appropriate, converts the amplified first vibration sensor signal into a digital signal with an AD converter, and outputs the digital signal to a signal processing unit 6a.

The second vibration sensor 32a outputs a second vibration sensor signal corresponding to a detected vibration in a triaxial direction to a second-vibration-sensor-signal reception unit 52a.

The second-vibration-sensor-signal reception unit 52a receives the second vibration sensor signal corresponding to the vibration in the triaxial direction from the second vibration sensor 32a, amplifies the second vibration sensor signal as appropriate, converts the amplified second vibration sensor signal into a digital signal with an AD converter, and outputs the digital signal to the signal processing unit 6a.

A vibration estimation unit 7a of the signal processing unit 6a according to the second embodiment includes a first vibration displacement rotation unit 73 that rotates the direction of the vibration detected by the first vibration sensor 31a and a second vibration displacement rotation unit 74 that rotates the direction of the vibration detected by the second vibration sensor 32a.

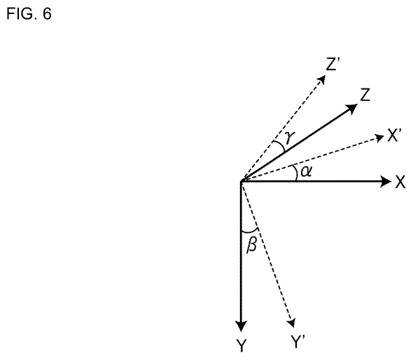

With reference to the displacement direction (X, Y, Z) of a body surface displacement signal obtained by the radio wave sensor 2, a vibration (x′, y′, z′) in the triaxial direction detected by the first vibration sensor 31a or the second vibration sensor 32a can be converted into a vibration direction (x, y, z) of the radio wave sensor 2 by using, for example, a coordinate rotation formula (1) below. In the coordinate rotation formula (1), α indicates a rotation angle in the X-axis direction, β indicates a rotation angle in the Y-axis direction, and γ indicates a rotation angle in the Z-axis direction. FIG. 6 is a diagram for describing an example of a coordinate rotation method according to the second embodiment.

$$\begin{matrix}[ Math . 1 ] & \\ [ \begin{matrix}x \\ y \\ z\end{matrix} ] = [ \begin{matrix}cos γ & sin γ & 0 \\ - s in γ & cos γ & 0 \\ 0 & 0 & 1\end{matrix} ] [ \begin{matrix}cos β & 0 & - sin β \\ 0 & 1 & 0 \\ sin β & 0 & cos β\end{matrix} ] [ \begin{matrix}1 & 0 & 0 \\ ( ) & cos α & sin α \\ 0 & - sin α & cos α\end{matrix} ] [ \begin{matrix}{x}^{′} \\ {y}^{′} \\ {z}^{′}\end{matrix} ] & ( 1 )\end{matrix}$$

The first vibration displacement rotation unit 73 outputs a first vibration signal that is rotated with reference to the displacement direction (X, Y, Z) of the body surface displacement signal, which is obtained by the radio wave sensor 2, by using, for example, the coordinate rotation formula (1). The second vibration displacement rotation unit 74 outputs a second vibration signal that is rotated with reference to the displacement direction (X, Y, Z) of the body surface displacement signal, which is obtained by the radio wave sensor 2, by using, for example, the coordinate rotation formula (1). Then, the vibration estimation unit 7a outputs, to the vibration removal unit 8, an estimated vibration signal corresponding to an estimated vibration component obtained by subtracting the first vibration signal output from the first vibration displacement rotation unit 73 from the second vibration signal output from the second vibration displacement rotation unit 74.

This makes it possible to align the displacement direction of the body surface displacement signal obtained by the radio wave sensor 2 with the vibration direction (displacement direction) of the estimated vibration signal calculated by the vibration estimation unit 7a.

The coordinate rotation formula (1) is an example, and the present disclosure is not limited to this example. Any coordinate rotation formula different from the coordinate rotation formula (1) may also be used to rotate the triaxial vibration direction detected by the first vibration sensor 31a or the second vibration sensor 32a.

According to the biological information detection device 1a of the second embodiment, the vibration direction of the second vibration component, which is obtained by the second vibration sensor 32a and includes many triaxial vibration components resulting from vehicle body vibrations in addition to the body movement component of the subject 4, and the vibration direction of the first vibration component, which is obtained by the first vibration sensor 31 and includes many triaxial vibration components resulting from vehicle body vibrations, are aligned with the displacement direction of the body surface displacement signal obtained by the radio wave sensor 2. Then, the first vibration signal aligned with the displacement direction of the body surface displacement signal is subtracted from the second vibration signal aligned with the displacement direction of the body surface displacement signal. As a result, an estimated vibration signal with reduced influence of vibration components resulting from vehicle body vibrations is obtained. Then, the estimated vibration signal, which is aligned with the displacement direction of the body surface displacement signal obtained by the radio wave sensor 2, is separated from the body surface displacement signal to generate a human biosignal in which the body movement component of the subject 4 is suppressed. Similarly to the first embodiment, this makes it possible to obtain accurate biological information.

In the example described in the second embodiment, the first vibration signal and the second vibration signal are generated by rotating them with reference to the displacement direction of the body surface displacement signal obtained by the radio wave sensor 2. Alternatively, the first vibration signal or the second vibration signal may be generated by rotating it with reference to the displacement direction of the body surface displacement signal obtained by the radio wave sensor 2.

### Third Embodiment

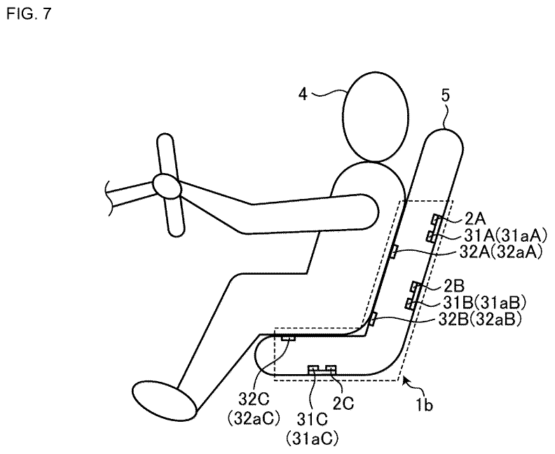

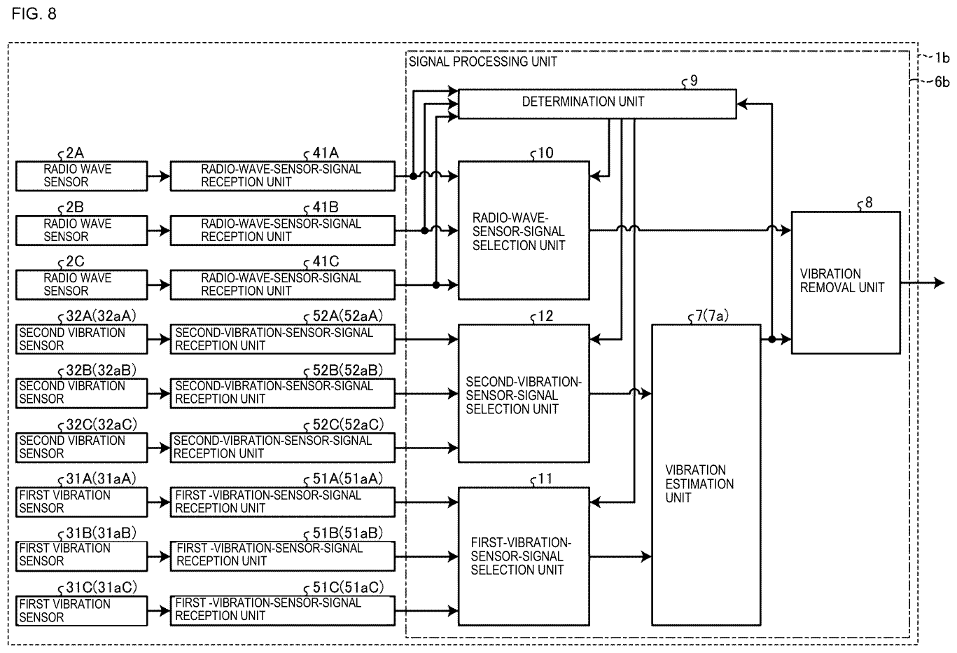

FIG. 7 is a side view of a biological information detection device according to a third embodiment, which is used for a driver monitoring system of a vehicle. FIG. 8 is a block diagram illustrating a schematic configuration of the biological information detection device according to the third embodiment.

A biological information detection device 1b according to the third embodiment includes multiple radio wave sensors 2, multiple first vibration sensors 31, and multiple second vibration sensors 32. In the example illustrated in FIGS. 7 and 8, the biological information detection device 1b includes radio wave sensors 2A, 2B, and 2C, first vibration sensors 31A, 31B, and 31C, and second vibration sensors 32A, 32B, and 32C. Each of the first vibration sensors 31A, 31B, and 31C corresponds to the first vibration sensor 31 according to the first embodiment. Each of the second vibration sensors 32A, 32B, and 32C corresponds to the second vibration sensor 32 according to the first embodiment. As an alternative configuration, the biological information detection device 1b may include first vibration sensors 31aA, 31aB, and 31aC, each of which corresponds to the first vibration sensor 31a according to the second embodiment, instead of the first vibration sensors 31A, 31B, and 31C and may include second vibration sensors 32aA, 32aB, and 32aC, each of which corresponds to the second vibration sensor 32a according to the second embodiment, instead of the second vibration sensors 32A, 32B, and 32C.

Also, the biological information detection device 1b according to the third embodiment includes multiple radio-wave-sensor-signal reception units 41, multiple first-vibration-sensor-signal reception units 51, and multiple second-vibration-sensor-signal reception units 52 that correspond to the radio wave sensors 2, the first vibration sensors 31, and the second vibration sensors 32, respectively. In the example illustrated in FIG. 8, the biological information detection device 1b includes radio-wave-sensor-signal reception units 41A, 41B, and 41C, first-vibration-sensor-signal reception units 51A, 51B, and 51C, and second-vibration-sensor-signal reception units 52A, 52B, and 52C. Each of the first-vibration-sensor-signal reception units 51A, 51B, and 51C corresponds to the first-vibration-sensor-signal reception unit 51 according to the first embodiment. Each of the second-vibration-sensor-signal reception units 52A, 52B, and 52C corresponds to the second-vibration-sensor-signal reception unit 52 according to the first embodiment. As an alternative configuration, the biological information detection device 1b may include first-vibration-sensor-signal reception units 51aA, 51aB, and 51aC, each of which corresponds to the first-vibration-sensor-signal reception unit 51a according to the second embodiment, instead of the first-vibration-sensor-signal reception units 51A, 51B, and 51C and may include second-vibration-sensor-signal reception units 52aA, 52aB, and 52aC, each of which corresponds to the second-vibration-sensor-signal reception unit 52a according to the second embodiment, instead of the second-vibration-sensor-signal reception units 52A, 52B, and 52C.

The signal processing unit 6b is formed on a dielectric substrate on which one of the radio wave sensors 2A, 2B, and 2C is provided. In the biological information detection device 1b according to the third embodiment, as illustrated in FIG. 8, the signal processing unit 6b includes a determination unit 9, a radio-wave-sensor-signal selection unit 10, a first-vibration-sensor-signal selection unit 11, and a second-vibration-sensor-signal selection unit 12 in addition to the components described in the first embodiment (or the second embodiment). The signal processing unit 6b may instead be formed on a substrate different from the dielectric substrates on which the radio wave sensors 2A, 2B, and 2C are provided.

In the configuration of the biological information detection device 1b of the third embodiment described above, the determination unit 9 is a signal control unit that controls the radio-wave-sensor-signal selection unit 10, the first-vibration-sensor-signal selection unit 11, and the second-vibration-sensor-signal selection unit 12 to select one of the radio wave sensors 2A, 2B, and 2C, one of the first vibration sensors 31A, 31B, and 31C, and one of the second vibration sensors 32A, 32B, and 32C, respectively.

The radio-wave-sensor-signal selection unit 10 outputs a body surface displacement signal selected by the determination unit 9 to the vibration removal unit 8.

The first-vibration-sensor-signal selection unit 11 outputs a first vibration sensor signal selected by the determination unit 9 to the vibration estimation unit 7 (7a).

The second-vibration-sensor-signal selection unit 12 outputs a second vibration sensor signal selected by the determination unit 9 to the vibration estimation unit 7 (7a).

In the example illustrated in FIGS. 7 and 8, it is assumed that the biological information detection device 1b includes three radio wave sensors 2, three first vibration sensors 31, and three second vibration sensors 32. However, each of the number of radio wave sensors 2, the number of first vibration sensors 31, and the number of second vibration sensors 32 is not limited to three. Each of the number of radio wave sensors 2, the number of first vibration sensors 31, and the number of second vibration sensors 32 may be two, four, or more. Also, the number of radio wave sensors 2, the number of first vibration sensors 31, and the number of second vibration sensors 32 are not necessarily the same.

The intensity of the body surface displacement signal, which is obtained by each radio wave sensor 2, varies depending on the position on the body of the subject 4 on which the electromagnetic wave emitted by the radio wave sensor 2 is incident. Specifically, the optimum electromagnetic wave incident position differs between a taller subject and a shorter subject. In other words, the strength of the body surface displacement signal obtained by each of the radio wave sensors 2A, 2B, and 2C varies depending on the sitting position of the subject 4 on the seat 5.

For example, the determination unit 9 compares body surface displacement signals obtained by the radio wave sensors 2A, 2B, and 2C, selects a body surface displacement signal with the highest reception intensity (received power), selects a first vibration sensor signal obtained by a first vibration sensor 31 (31a) disposed on the same dielectric substrate as a radio wave sensor 2 that has obtained the selected body surface displacement signal, and selects a second vibration sensor signal obtained by a second vibration sensor 32 (32a) that is disposed relatively close to the first vibration sensor 31 (31a) that has obtained the selected first vibration sensor signal.

As another example, the determination unit 9 may be configured to determine the correlation between the selected radio wave sensor signal and the estimated vibration signal output from the vibration estimation unit 7 (7a) and reselect one or both of the first vibration sensor signal and the second vibration sensor signal. Specifically, the determination unit 9 calculates a correlation coefficient between the selected radio wave sensor signal and the estimated vibration signal output from the vibration estimation unit 7 (7a), reselects one or both of the first vibration sensor signal and the second vibration sensor signal assuming the calculated correlation coefficient is less than a predetermined threshold, and performs a similar correlation coefficient calculation process. Then, the determination unit 9 selects a combination of a first vibration sensor signal and a second vibration sensor signal with which the correlation coefficient becomes greater than or equal to the predetermined threshold.

The biological information detection device 1b of the third embodiment includes multiple radio wave sensors 2, multiple first vibration sensors 31, and multiple second vibration sensors 32. With this configuration, the optimum body surface displacement signal, the optimum first vibration sensor signal, and the optimum second vibration sensor signal are selected according to, for example, the height and physique or the driving position of the subject 4. Then, an estimated vibration signal obtained from the selected first vibration sensor signal and the selected second vibration sensor signal is separated from the selected body surface displacement signal to generate a human biosignal in which the body movement component of the subject 4 is suppressed. This makes it possible to obtain accurate biological information by suppressing variations resulting from the height and physique or the driving position of the subject 4.

The above-described embodiments are intended to facilitate the understanding of the present disclosure and are not intended to limit the interpretation of the present disclosure. The present disclosure may be modified or improved without departing from the spirit of the present disclosure and may include equivalents thereof.

As described above or alternatively, the present disclosure may provide configurations as described below.

(1) A biological information detection device according to an aspect of the present disclosure includes a radio wave sensor that detects a body surface displacement of a human body; a first vibration sensor that is disposed together with the radio wave sensor at a position away from a supporting surface that supports the human body; a second vibration sensor that is disposed closer to the supporting surface than the first vibration sensor; and a signal processing unit that generates a biosignal of the human body based on the body surface displacement obtained by the radio wave sensor, a first vibration component obtained by the first vibration sensor, and a second vibration component obtained by the second vibration sensor.

With this configuration, the biosignal of the human body is generated based on the body surface displacement obtained by the radio wave sensor, the first vibration component obtained by the first vibration sensor, and the second vibration component obtained by the second vibration sensor. This configuration makes it possible to suppress both the influence of a body movement component of a subject and the influence of vibration components resulting from vibrations of the body of a vehicle (e.g., an automobile).

(2) In the biological information detection device described in (1), the signal processing unit includes a vibration estimation unit that subtracts the first vibration component from the second vibration component to generate an estimated vibration component and a vibration removal unit that separates the estimated vibration component from the body surface displacement.

In this configuration, the first vibration component, which includes many vibration components resulting from vehicle body vibrations, is subtracted from the second vibration component, which includes vibration components resulting from the vehicle body vibrations. This makes it possible to obtain an estimated vibration component with reduced influence of vibration components resulting from, for example, vehicle body vibrations. Then, the estimated vibration component can be separated from the body surface displacement obtained by the radio wave sensor.

(3) In the biological information detection device described in (2), the vibration removal unit generates the biosignal using an adaptive filter.

This configuration makes it possible to generate a human biosignal in which the body movement component of a subject is suppressed. This in turn makes it possible to obtain accurate biological information.

(4) In the biological information detection device described in (2), the vibration estimation unit rotates at least one of the first vibration component and the second vibration component.

This configuration makes it possible to align the displacement direction of the body surface displacement obtained by the radio wave sensor with the vibration direction (displacement direction) of the estimated vibration component calculated by the vibration estimation unit.

(5) In the biological information detection device described in (4), the vibration removal unit generates the biosignal using an adaptive filter.

This configuration makes it possible to generate a human biosignal in which the body movement component of a subject is suppressed. This in turn makes it possible to obtain accurate biological information.

(6) In the biological information detection device described in (2) to (5), at least one of the radio wave sensor, the first vibration sensor, and the second vibration sensor comprises multiple radio wave sensors, multiple first vibration sensors, or multiple second vibration sensors; and the signal processing unit appropriately selects a combination of the body surface displacement, the first vibration component, and the second vibration component upon generating the biosignal.

This configuration makes it possible to select at least one of the optimum body surface displacement, the optimum first vibration component, and the optimum second vibration component from multiple body surface displacements, multiple first vibration components, or multiple second vibration components based on the height and physique or the driving position of the subject. This makes it possible to obtain accurate biological information by suppressing variations resulting from the height and physique or the driving position of the subject.

(7) In the biological information detection device described in (6), the radio wave sensor may comprise multiple radio wave sensors, and the signal processing unit may include a radio-wave-sensor-signal selection unit that selects one of body surface displacements obtained by the multiple radio wave sensors.

(8) In the biological information detection device described in (6), the first vibration sensor may comprise multiple first vibration sensors, and the signal processing unit may include a first-vibration-sensor-signal selection unit that selects one of first vibration components obtained by the multiple first vibration sensors.

(9) In the biological information detection device described in (6), the second vibration sensor may comprise multiple second vibration sensors, and the signal processing unit may include a second-vibration-sensor-signal selection unit that selects one of second vibration components obtained by the multiple second vibration sensors.

(10) A vehicle according to an aspect of the present disclosure includes the biological information detection device described in (1) to (9).

This configuration makes it possible to realize a vehicle including a biological information detection device that is capable of suppressing both the influence of a body movement component of a subject and the influence of vibration components resulting from vibrations of the body of a vehicle and thereby obtaining accurate biological information.

The present disclosure makes it possible to realize a biological information detection device capable of obtaining accurate biological information and a vehicle including the biological information detection device.

### REFERENCE SIGNS LIST

1, 1a, 1b biological information detection device
2 radio wave sensor
3 housing
4 subject
5 seat
5a internal material
5b surface material
6, 6a signal processing unit
7, 7a vibration estimation unit
8 vibration removal unit
8a filter
8b adaptive processing unit
9 determination unit
10 radio-wave-sensor-signal selection unit
11 first-vibration-sensor-signal selection unit
12 second-vibration-sensor-signal selection unit
31, 31A, 31B, 31C, 31a, 31aA, 31aB, 31aC first vibration sensor
32, 32A, 32B, 32C, 32a, 32aA, 32aB, 32aC second vibration sensor
41, 41A, 41B, 41C radio-wave-sensor-signal reception unit
51, 51A, 51B, 51C, 51a, 51aA, 51aB, 51aC first-vibration-sensor-signal reception unit
52, 52A, 52B, 52C, 52a, 52aA, 52aB, 52aC second-vibration-sensor-signal reception unit
71 first displacement conversion unit
72 second displacement conversion unit
73 first vibration displacement rotation unit
74 second vibration displacement rotation unit

1. A biological information detection device comprising:
a radio wave sensor that detects a body surface displacement of a human body;
a first vibration sensor that is disposed together with the radio wave sensor at a position away from a supporting surface that supports the human body;
a second vibration sensor that is disposed closer to the supporting surface than the first vibration sensor; and
circuitry configured to generate a biosignal of the human body based on the body surface displacement obtained by the radio wave sensor, a first vibration component obtained by the first vibration sensor, and a second vibration component obtained by the second vibration sensor.

2. The biological information detection device according to claim 1, wherein
the circuitry is further configured to implement
a vibration estimation unit that subtracts the first vibration component from the second vibration component to generate an estimated vibration component, and
a vibration removal unit that separates the estimated vibration component from the body surface displacement.

3. The biological information detection device according to claim 2, wherein the vibration removal unit generates the biosignal using an adaptive filter.

4. The biological information detection device according to claim 2, wherein the vibration estimation unit rotates at least one of the first vibration component and the second vibration component.

5. The biological information detection device according to claim 4, wherein the vibration removal unit generates the biosignal using an adaptive filter.

6. The biological information detection device according to claim 5, wherein
at least one of the radio wave sensor, the first vibration sensor, and the second vibration sensor comprises multiple radio wave sensors, multiple first vibration sensors, or multiple second vibration sensors; and
the circuitry appropriately selects a combination of the body surface displacement, the first vibration component, and the second vibration component upon generating the biosignal.

7. The biological information detection device according to claim 6, wherein
the radio wave sensor comprises multiple radio wave sensors; and
the circuitry is further configured to select one of body surface displacements obtained by the multiple radio wave sensors.

8. The biological information detection device according to claim 6, wherein
the first vibration sensor comprises multiple first vibration sensors; and
the circuitry is further configured to select one of first vibration components obtained by the multiple first vibration sensors.

9. The biological information detection device according to claim 6, wherein
the second vibration sensor comprises multiple second vibration sensors; and
the circuitry is further configured to select one of second vibration components obtained by the multiple second vibration sensors.

10. A vehicle comprising the biological information detection device according to claim 9.

11. The biological information detection device according to claim 2, wherein
at least one of the radio wave sensor, the first vibration sensor, and the second vibration sensor comprises multiple radio wave sensors, multiple first vibration sensors, or multiple second vibration sensors; and
the circuitry is further configured to appropriately select a combination of the body surface displacement, the first vibration component, and the second vibration component upon generating the biosignal.

12. The biological information detection device according to claim 11, wherein
the radio wave sensor comprises multiple radio wave sensors; and
the signal processing unit includes a radio-wave-sensor-signal selection unit that selects one of body surface displacements obtained by the multiple radio wave sensors.

13. The biological information detection device according to claim 11, wherein
the first vibration sensor comprises multiple first vibration sensors; and
the circuitry is further configured to select one of first vibration components obtained by the multiple first vibration sensors.

14. The biological information detection device according to claim 11, wherein
the second vibration sensor comprises multiple second vibration sensors; and
the circuitry is further configured to select one of second vibration components obtained by the multiple second vibration sensors.

15. The biological information detection device according to claim 3, wherein
at least one of the radio wave sensor, the first vibration sensor, and the second vibration sensor comprises multiple radio wave sensors, multiple first vibration sensors, or multiple second vibration sensors; and
the circuitry is further configured to select a combination of the body surface displacement, the first vibration component, and the second vibration component upon generating the biosignal.

16. The biological information detection device according to claim 15, wherein
the radio wave sensor comprises multiple radio wave sensors; and
the circuitry is further configured to select one of body surface displacements obtained by the multiple radio wave sensors.

17. The biological information detection device according to claim 15, wherein
the first vibration sensor comprises multiple first vibration sensors; and
the circuitry is further configured to select one of first vibration components obtained by the multiple first vibration sensors.

18. The biological information detection device according to claim 15, wherein
the second vibration sensor comprises multiple second vibration sensors; and
the circuitry is further configured to select one of second vibration components obtained by the multiple second vibration sensors.

19. The biological information detection device according to claim 4, wherein
at least one of the radio wave sensor, the first vibration sensor, and the second vibration sensor comprises multiple radio wave sensors, multiple first vibration sensors, or multiple second vibration sensors; and
the circuitry is further configured to select a combination of the body surface displacement, the first vibration component, and the second vibration component upon generating the biosignal.

20. The biological information detection device according to claim 19, wherein
the radio wave sensor comprises multiple radio wave sensors; and
the circuitry is further configured to select one of body surface displacements obtained by the multiple radio wave sensors,
wherein
the first vibration sensor comprises multiple first vibration sensors; and
the circuitry is further configured to select one of first vibration components obtained by the multiple first vibration sensors, and
wherein
the second vibration sensor comprises multiple second vibration sensors; and
the circuitry is further configured to select one of second vibration components obtained by the multiple second vibration sensors.
